(19) United States
(12) Patent Application Publication
(10) Pub. No.: US 2024/0008745 A1
(43) Pub. Date: Jan. 11, 2024

(54) Hand-Held Device for Fluorescence Excitation and for Irradiating Microorganisms in the Mouth and Throat

(71) Applicant: Karsten KÖNIG, Berlin (DE)

(72) Inventor: Karsten KÖNIG, Berlin (DE)

(21) Appl. No.: 18/349,905

(22) Filed: Jul. 10, 2023

Related U.S. Application Data
(63) Continuation of application No. 17/421,703, filed on Jul. 8, 2021.

(30) Foreign Application Priority Data
Jan. 8, 2019 (DE) 102019100295.8

(51) Int. Cl. A61B 5/00; A46B 7/04; A46B 9/04; A46B 15/00; A61C 17/22; A61C 17/26; A61C 17/34

(52) U.S. Cl. CPC A61B 5/0071; A46B 7/04; A46B 9/04; A46B 15/0034; A46B 15/0036; A61B 5/0088; A61C 17/224; A61C 17/26; A61C 17/34

(57) ABSTRACT

The invention relates to a hand-held device for excitation and irradiation of pathogenic microorganisms in the mouth and throat, e.g. a toothbrush comprising at least one excitation light source in the short-wave visible spectral range for auto-fluorescence excitation of the pathogenic microorganisms, at least one primary irradiation light source in the red spectral range for primary irradiation of the pathogenic microorganisms and for transillumination, and optionally at least one secondary irradiation light source in the visible spectral range for secondary irradiation of the pathogenic microorganisms, wherein the irradiation light sources have spectral components that can be absorbed by endogenous porphyrins, which are produced by the pathogenic microorganisms, whereby a fluorescence excitation and an inactivation of the pathogenic microorganisms occurs on the basis of subsequent processes. In order to prevent the unintentional irradiation of the eyes, a pressure sensor is designed to release higher light intensities only once a contact pressure has been measured. In addition, the radiation must leave the hand-held device in a divergent manner. The spatially resolved detection of the fluorescence of the pathogenic microorganisms can optionally be used to induce the inactivation of the bacteria by targeted irradiation in the fluorescent range.

### PRIORITY DATA

This application is a continuation of U.S. patent application Ser. No. 17/421,703, filed on Jul. 8, 2021, which is the national phase filing under 35 U.S.C. 371 of PCT Application Number PCT/EP2020/050222, filed Jan. 7, 2020, which claims the benefit of German Patent Application Serial Number 102019100295.8, filed on Jan. 8, 2019, each of which are incorporated herein by reference.

The invention relates to a hand-held device, for example in the form of a toothbrush, for fluorescence excitation and irradiation of pathogenic microorganisms in the mouth and throat by means of optical radiation, which is suitable for visualizing the occurrence of these microorganisms, such as bacteria, which play a decisive role in the pathogenesis of dental caries, gingivitis and periodontitis, but also in inflammatory changes in the throat, such as sinusitis, and slowing down or completely preventing propagation of these microorganisms by inactivating them, as well as killing the microorganisms already existing.

More specifically, the invention relates to a handheld device for fluorescence excitation and irradiation of pathogenic microorganisms in the mouth and throat, such as a toothbrush, comprising at least one excitation light source in the short-wave visible spectral range for auto-fluorescence excitation and irradiation of the pathogenic microorganisms at the surface of the region to be targeted in the mouth and throat, at least one primary irradiation light source in the red spectral range for primary irradiation of the pathogenic microorganisms and for transillumination, and optionally at least one secondary irradiation light source in the visible spectral range between 450 nm and 600 nm for secondary irradiation of the pathogenic microorganisms, wherein the irradiation light sources have spectral components that can be absorbed by endogenous porphyrins which are produced by the pathogenic microorganisms, whereby a fluorescence excitation and an inactivation of the pathogenic microorganisms occurs on the basis of subsequent processes. In order to prevent the unintentional irradiation of the eyes, the hand-held device also comprises a pressure sensor designed to release higher light intensities only once a contact pressure has been measured. In addition, the handheld device is arranged such that the radiation leaves the handheld device as a divergent beam. The spatially resolved detection of the fluorescence of the pathogenic microorganisms can also be used to induce the inactivation of the bacteria by targeted irradiation in the fluorescent range.

In the field of dentistry, oral and maxillofacial medicine, a wide variety of techniques and devices have been used for some time to detect the health condition of a patient's teeth, for example a bacterial infestation directly on plaque-free tooth surfaces or bacterial occurrences in plaque layers in varying depths, by visual examination or by using X-rays, and taking appropriate countermeasures based thereon. Such bacterial infestations in the throat can cause serious systemic diseases when transmitted into the blood circulation. For example, clinical studies have shown an almost twofold increase in the risk of heart disease and an almost threefold increase in the risk of stroke in people suffering from periodontitis. Accordingly, effective countermeasures are of great importance. Known countermeasures include daily dental hygiene by flossing, brushing and mouth rinsing, as well as tooth cleaning by a specialist, such as a dentist, wherein use may also be made of ultrasonic cleaning procedures. Usually, however, only partly sufficient plaque removal is achieved. For example, conventional mechanical tooth cleaning using a toothbrush only reaches one third of the tooth surface. In most refracrory cases, antibiotically active pharmaceuticals are often made use of as a bacteria-inactivating measure. However, as antibiotic-resistance of bacterial strains is currently increasing, the search for more efficient diagnostic and treatment approaches to microbial teeth infections is gaining essential importance.

In recent years, antibacterial effect of irradiating tooth surfaces using high intensity laser light has already been studied in more detail. This process of bacterial damage is essentially subject to a photothermal effect, whereby ablative bacterial removal can additionally be achieved by the ablative effect of the laser. The obvious disadvantages of this type of treatment are the usually complex and large laser systems required for this purpose, as well as the high light intensities required, ranging from several kilowatts per square centimeter (kW/cm²) to over one gigawatt per square centimeter (GW/cm²). Inactivation by light-induced chemical reactions involving light-sensitive substances, which usually are to be externally added to the microorganisms, has been found to be another form of treatment for removing pathogenic microorganisms. However, the aforementioned types of therapy not only require skilled professionals and appropriate clinical equipment, but are also unsuitable for the daily routine at home.

With the emerging use of laser-assisted autofluorescence spectroscopy in clinical diagnosis, it has recently been increasingly recognized that a wide variety of bacterial species naturally synthesize fluorescent substances and, when stimulated appropriately, provide a distinctive photoelectric effect through autofluorescence that can be used to diagnose bacterial infestation of organs and tissues. Based on these findings in clinical practice, there was the need to further develop the present invention to be implemented in the medical-technical field of dentistry, oral and maxillofacial medicine, and to develop suitable new medical-technical devices which, on the one hand, are capable of determining the teeth health condition, especially the presence of caries, plaque or bacterial infestation on teeth, and, on the other hand, in the case of any infestation determination being positive, performing corresponding treatment in a simple manner without the disadvantages of requiring clinical practice, i.e. without the need for skilled persons. i.e. without the requirement of skilled persons or costly and large laser systems for appropriately high light intensities.

Therefore, for example, in the publication DE 30 31 249 C2 or also in the publication DE 93 17 984 U1, non-contact diagnostic methods for detecting caries, plaque or bacterial infestation on teeth have been proposed, wherein a tooth is irradiated with an almost monochromatic light source. Due to the irradiation of the tooth with monochromatic light, fluorescence radiation is excited at the tooth, wherein the fluorescence spectrum shows distinct differences between healthy and diseased tooth areas. By detecting and evaluating the fluorescence spectrum of the tooth irradiated in this way, a healthy tooth area can thus be clearly distinguished from a diseased tooth area.

Inactivation of microorganisms by means of ultraviolet (UV) radiation is also broadly used, for example in the publication EP 0 818 181 A1, in which the use of UV laser radiation for caries removal is proposed. However, the disadvantages of UV radiation, besides low penetration depth of the radiation of a few micrometers, especially are the capability of UV radiation to induce carcinogenic processes.

Inactivation of bacteria by visible radiation using certain exogenous light-sensitive substances, so-called photosensitizers, is also known in the field of dentistry and oral and maxillofacial medicine. When appropriately exciting such photosensitizers using light, oxygen radicals and electronically excited oxygen, so-called singlet oxygen, is formed as a result of photooxidation processes, which have a cell-destructive effect on biological matter in subsequent reactions. The photosensitizers are used in the course of the so-called photodynamic therapy (PDT), especially in tumor therapy ((e.g. Cancer Res. 38(1978) p. 2628-2635). In the related literature (e.g. J. Photochem. Photobiol.B 21(1993) pp. 81-86) it has been described that addition of photosensitizers to bacterial cultures and subsequent light activation may result in inactivation of the bacteria. However, the described photodynamic therapy of tissues and the photodynamic inactivation of microorganisms have the decisive disadvantage that addition of photosensitizers is required. In order to avoid high damage to surrounding tissue during irradiation, enrichment of the photosensitizers in the target is required. However, this is not realizable in general and thus is the subject of extensive researches. With exogenous photosensitizers clinically used to date, enrichment outside the target material occurs, e.g. in healthy skin, involving the risk of induced temporary photosensitivity of the patient and undesirable photodamage of the healthy tissue.

As an example of the results of the previously mentioned researches, a laser arrangement in connection with light guides can be seen in EP 0 743 029 A2, based on photodynamic inactivation of pathogenic microorganisms in the mouth and throat, using exogenous photosensitizers, wherein application of the photosensitizer is to be performed by addition thereof to a toothpaste or a liquid. The disadvantages of this arrangement, in addition to the use of exogenous photosensitizers, are the use of laser light guiding systems and the high safety requirements correspondingly required due to the use of laser radiation in the face area. In addition, administration of a toothpaste or liquid containing the photosensitizer also involves the risk of accumulating the photosensitizer in the healthy mucosa. Irradiation of the mucosa enriched with the photosensitizer, especially in the dental area and the tongue, can cause substantial tissue damage.

Furthermore, it is known that respective bacteria in the throat can also cause inflammatory conditions, for example during inflammation of the paranasal sinuses, also known as sinusitis, including sinusitis maxillaris, also known as maxillary sinusitis, which regularly affects a high percentage of the European population due to viral and bacterial infections. In Germany, for example, every year approximately 15% of the population is affected by these disease conditions. Due to swelling of the mucous membranes, secretions can no longer drain properly. If the condition persists for more than one week, bacteria are usually involved. If the condition persists for more than two months, it is called chronic sinusitis. Up to now, diagnosis of sinusitis has been based on examination of nasal secretions, or has been based on computer tomography (CT) or magnetic resonance imaging (MRI), which, however, are expensive and time-consuming procedures and are not very suitable especially for children and adolescents because of the radiation exposure. However, in an optical manner, examinations of the throat can also be performed endoscopically. Another optical method was published by Wang et al. in 2005, utilizing a near-infrared (NIR) light source in the throat and a NIR-sensitive CCD camera that detects the transilluminated NIR radiation. Fluid accumulation due to sinusitis results in altered transillumination (Wang et al. Near infrared transillumination of the maxillary sinuses: overview of methods and preliminary clinical results. Proceed. SPIE 5686 (2005)). The disadvantage of this arrangement is the use of non-visible NIR light sources and NIR-sensitive CCD special cameras, wherein it should be noted that most CCD cameras are equipped with NIR-blocking filters that prevent detection of NIR radiation.

Accordingly, the task of the present invention is to provide a hand-held device in the form of a toothbrush or a so-called light toothbrush for transillumination and photodynamic inactivation of pathogenic microorganisms in the mouth and throat, which is easy to handle in use at home and is without risk to a user and which does not require the use of exogenous photosensitizers. Herein, high penetration depth of the radiation into the affected tissue as well as dosability of the radiation intensity are advantageous, among other things.

From our own research in this regard, when developing the present invention, it was found that certain pathogenic microorganisms, which especially are present in the mouth and throat, intrinsically synthesize the metal-free, fluorescent and light-sensitive porphyrins protoporphyrin IX (PP EX) and coproporphyrin (CP), among other things (Cell Mol. Biol. 46(2000) pp. 1297-1303). Such microorganisms include, for example, the pathogenic Gram-positive porphyrin-producing ATCC strains Propionibacterium acnes, Actinomyces odontolyticus, and Porphyromonas gingivalis, as well as Prevotella species, which may play a significant role in the pathogenesis and expression of caries, ginigvitis, and periodontitis, and which synthesize the aforementioned porphyrins without artificial external stimulation. Accordingly, these microorganisms as carriers of endogenous photosensitizers are sensitive to visible light. In additional research in this field, fluorescence studies revealed that protoporphyrin IX and coproporphyrin accumulate in decayed teeth as well as in dental plaque (Cell Mol. Biol. 44(1998) pp. 1293-1300).

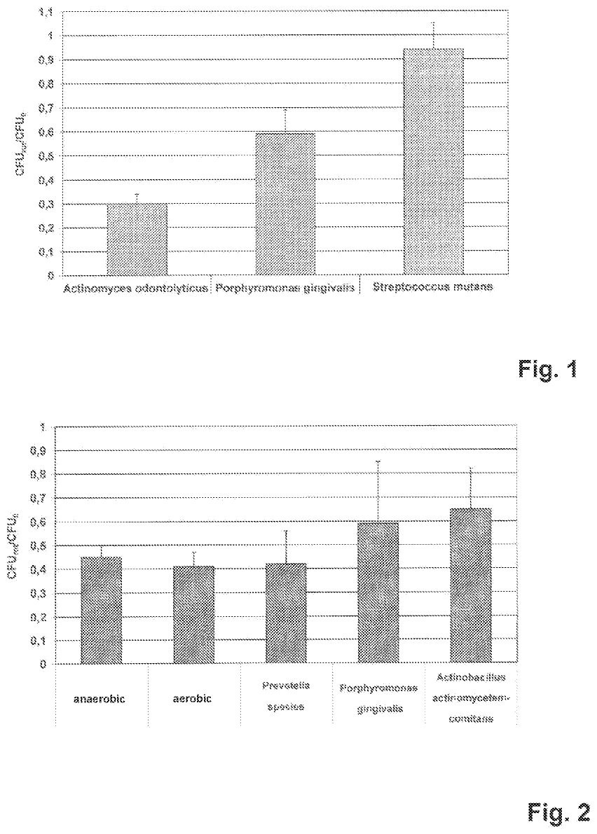

In the research conducted when developing the present invention, it was surprisingly found that inactivation of the aforementioned pathogenic Gram-positive porphyrin-producing ATCC strains is possible by irradiation in the red spectral region around 633 nm, and with very low and thus tissue-harmless irradiation intensities in the range of milliwatts per square centimeter (mW/cm²), without the need of using any exogenous photosensitizer. Exemplary results of this own research can be seen in FIGS. 1 and 2, in which the quotient of the number of colony forming units (CFU) of irradiated samples (“CFUrot”) vs. non-irradiated samples (“CFUO”) is shown, wherein this quotient corresponds to the survival rate. For these experiments, cultures of large-scale grown bacteria as well as microbiological samples from periodontitis patients were irradiated once using radiation having a wavelength of approximately 633 nm, for example 632.8 nm, at 100 mW/cm² of intensity and 360 J/cm² of energy density, for example using a helium-neon laser. A distinctive inactivating effect was observed when using only a single irradiation. As can be seen from FIGS. 1 and 2, the mean survival rate for the porphyrin-containing bacterial strains Actinomyces odoniolyticus and Porphyromonas gingivivalis was only 30%±4% and 59%±10%, respectively. In contrast, the bacterial strain Streptococcus mutans, in which no detectable porphyrins could be detected, showed no, but significant inactivation. The survival rate for microbiological samples from periodontitis patients was 45% for anaerobic bacteria, 41% for aerobic bacteria, 42% for Prevotella species, 59% for Porphyromonas gingivalis and 65% for Actinobacillus actinomycetemcomitans. These results clearly demonstrate that irradiation of porphyrin-containing pathogenic microorganisms with radiation in the red spectral region results in effective inactivation of these bacteria without the use of exogenous photosensitizers.

Typically, activation of the photosensitizers accordingly occurs with radiation in the red spectral region, i.e., in the wavelength range from 630 to 700 nm. Such radiation in the red spectral range is located in the region of the so-called “optical window”, i.e. the region of relatively high penetration depth of radiation into biological tissue. The penetration depth, which can be defined as the tissue depth where the decrease in light intensity to about 37% has occurred, is typically one to five millimeters in biological tissues. A comparably high penetration depth can be achieved when irradiating in the yellow spectral range, i.e., in the wavelength range from 560 to 590 nm. In contrast, the penetration depth greater than 2 μm of ultraviolet, violet and infrared radiation in biological tissues is typically only in the micrometer range.

According to the invention, a hand-held device for the excitation and irradiation of pathogenic microorganisms in the mouth and throat is provided, which comprises at least one excitation light source in the short-wave visible spectral range for auto-fluorescence excitation and irradiation of the pathogenic microorganisms at the surface of the region to be irradiated. The short-wave visible spectral range mentioned herein preferably comprises violet light in a range from 400 nm to 410 nm. Typical light penetration depths in this spectral range are a few hundred micrometers in the tooth, i.e. <1 mm. Furthermore, the handheld device according to the invention comprises at least one primary irradiation light source in the red spectral range, i.e. having a wavelength around 630 nm, both for primary irradiation of the pathogenic microorganisms and for transillumination, i.e. having dual applicability. Furthermore, the handheld device according to the invention optionally comprises at least one secondary irradiation light source in the visible spectral range, preferably green or yellow, for irradiating the pathogenic microorganisms. The excitation light source, as well as the primary and the optional secondary irradiation light source emit appropriate visible radiation, wherein all irradiation light sources have different wavelengths, and the respective emitted radiation is comprised of spectral components which can be absorbed by endogenous porphyrins produced by the pathogenic microorganisms. Thus, fluorescence formation and inactivation of the pathogenic microorganisms can be achieved on the basis of subsequent processes. Besides direct irradiation of the microorganisms for inactivation thereof, radiation of the primary irradiation light source in the red spectral range around 630 nm is additionally also suitable for detecting inflammatory processes in the throat (sinusitis) by transillumination. In other words, the handheld device according to the invention comprises at least one violet radiation source (405 nm) and one red radiation source (633 nm). Herein, the violet radiation is used for fluorescence excitation and inactivation of bacteria on the surface of the dental hard tissue and soft tissue. On the one hand, the red radiation of the primary irradiation light source is for the already mentioned dual use applicability, for detection of sinusitis by the altered scattered radiation, which scattered radiation can be detected by transillumination through the skin in the face region, i.e. by those radiation components which are produced by a radiation source introduced into the mouth and which passes the skin and thus can be detected in the face region. In this case, the transillumination image of the pathological area shows a spatially altered and intensity-altered pattern compared to healthy areas. On the other hand, the red radiation also serves to inactivate bacteria in the tissue depth, i.e. in the deeper tissue layers of several millimeters, such as in deeper layers of the hard dental tissue. The hand-held device according to the invention also comprises a pressure sensor, the measurement output of which is used to increase an irradiation light intensity of the irradiation light sources to values in a range of from 10 mW/cm² to 100 mW/cm² when a contact pressure of the hand-held device onto the targeted surface area is detected, to achieve intensification of pathogenic microorganism inactivation. In this way, it can be ensured that no excessive irradiation intensity is emitted by the hand-held device according to the invention when the hand-held device is not located in the mouth and throat, especially to avoid irradiation of the eyes of a user with the increased irradiation intensity.

The pathogenic microorganisms, for example corresponding to the pathogenic Gram-positive porphyrin-producing ATCC strains Propionibacterium acnes, Actinomyces odontolyticus and Porphyromonas gingivalis as well as the Prevotella species. Based on the excitation light source, the present hand-held device according to the invention enables excitation of intrinsic fluorescence of these pathogenic microorganisms, so that a user can recognize with the naked eye where or on which teeth or tooth areas such pathogenic microorganisms are located. In addition, the excitation light source in the visible short-wave spectral range can already cause initial inactivation of the pathogenic microorganisms. Subsequently, in addition to the transillumination already described, the user is enabled, especially by using the primary and/or the optional secondary irradiation light source, to irradiate the intrinsically fluorescent and other pathogenic microorganisms using wavelengths that are absorbed by the endogenous porphyrins produced by the pathogenic microorganisms. By appropriate subsequent processes initiated by the absorption of this radiation, the microorganisms can be induced to further fluorescence formation, as well as to at least partial, preferably complete inactivation. Consequently, the hand-held device according to the invention is suitable for inhibiting the occurrence and propagation of certain pathogenic microorganisms and for destroying such microorganisms already existing by initiating photodynamic reactions involving endogenous porphyrins. Administration of any exogenous photosensitizer is not further required for this purpose.

According to a preferred embodiment, the excitation light source for auto-fluorescence excitation of the pathogenic microorganisms emits an excitation radiation corresponding to an absorption maximum of porphyrins, wherein the excitation radiation can, for example, be in a range from 400 nm to 410 nm, such as in the range of 405 nm, which corresponds to violet light. In this way, the maximum possible intrinsic fluorescence of the targeted microorganisms can be achieved to enable a user to detect as many microorganism colonies as possible on his tooth surfaces with maximum ease, and to fight them in a targeted manner. However, the radiation does not usually reach a deep penetration depth, so that surface microorganisms can increasingly be targeted and inactivated, due to the property of violet light low light penetration depth into biological soft and hard tissue.

According to another preferred embodiment of the present invention, radiation in the visible longer wavelength spectral range is also emitted by the handheld device according to the invention. In this case, the irradiation for example has an additional wavelength component in a range from 630 nm to 700 nm, preferably in a range from 630 nm to 635 nm, further preferably light in the range of 633 nm, which corresponds to red light. When radiation in the range of 630 nm to 640 nm is applied, a high penetration depth of the radiation as well as targeted irradiation into an absorption band of the endogenous porphyrins protoporphyrin IX and coproporphyrin is provided, whereby deeper microorganisms may be reached and inactivated. By successive irradiation using the radiation from the excitation light source followed by radiation from the primary irradiation light source having a wavelength component in a range from 630 nm to 700 nm, preferably in a range from 630 nm to 635 nm, further preferably in a range of 633 nm, and optionally also irradiation using the secondary irradiation light source having a wavelength component in a range from 490 nm to 560 nm (green-yellow), preferably in the range of 505 nm, irradiation of the desired area can be achieved in different penetration depths to achieve most extensive possible inactivation of the targeted microorganisms. Optionally, therefore, a third radiation source in the visible range, preferably having green or yellow light, can be used, which preferably is for inactivation of bacteria surface located and slightly deeper. The light sources may further also emit exclusively in the specified range, continuously or pulsed, but may alternatively emit radiation in a wider visible wavelength range, as well as provide a white light source, if desired. Cancer-causing effects from radiation in the above-mentioned irradiation wavelength ranges are not known. Short-wave visible radiation in the blue or violet spectral range, especially around 405 nm, and in the yellow or green spectral range, especially around 505 nm, can be used as the endogenous porphyrins have absorption bands therein. However, as the irradiation depth is lower than in the red spectral range, only microorganisms in the surface region can predominantly be inactivated by the short-wave visible radiation. When using radiation in the wavelength ranges mentioned above, application of exogenous photosensitizers for inactivating porphyrin-producing pathogenic microorganisms is not required, and the hand-held device according to the invention is capable of inactivating the targeted pathogenic microorganisms without further aids, such as exogenous photosensitizers, thereby eliminating potentially arising risks to the user, such as caries, ginigvitis and periodontitis. All radiation sources of the hand-held device according to the invention contain spectral components similar to those of endogenous porphyrins, thus being able to initiate fluorescence and photochemical processes for inactivation.

According to another preferred embodiment of the present invention, radiation emission of the excitation light source, the primary irradiation light source, and the secondary irradiation light source is performed in the manner of a traffic light circuit. Accordingly, the radiation of the excitation light source is emitted first, for example, at an upper end of the handheld device. Following detection of the intrinsically fluorescent microorganisms and irradiation of the microorganisms with the excitation light source in a surface region, radiation of the primary irradiation light source can subsequently be emitted, for example, located below the excitation light source. Radiation of the optional secondary irradiation light source can subsequently be performed, which secondary irradiation light source can be arranged, for example, below the primary irradiation light source. Accordingly, for example, irradiation of the teeth and the surrounding soft tissue of a user is first performed using excitation radiation at a position on the hand-held device, followed by irradiation of the microorganisms in the mouth and throat of the user using irradiation from the primary irradiation light source at a position located more inferior on the hand-held device, followed by irradiation of the microorganisms in the mouth and throat of the user using irradiation from the secondary irradiation light source at a position located still more inferior on the hand-held device. Accordingly, such a traffic light circuit of the various light sources of the hand-held device according to the present invention can fulfill not only a medical-technical purpose but, in addition, an aesthetic purpose, whereby passing through of the traffic light circuit steps also fulfills a control and controllability purpose regarding correct course of the irradiation.

According to another preferred embodiment of the present invention, the handheld device may further comprise at least one light detection device for detecting an intrinsic fluorescence radiation of the pathogenic microorganisms, so that the user is not required to visually detect all intrinsically fluorescent microorganisms, but may pass this task to the light detection device. Accordingly, the light detection device can detect the intrinsic fluorescence of the excited microorganisms and output a corresponding detection signal, for example based on a haptic or acoustic feedback to the user, wherein the strength of the feedback can reflect the intensity of the intrinsic fluorescence. Furthermore, the light detection device can output the detection signal, i.e. the distribution of the fluorescent microorganisms on the user's teeth, to the outside by means of a transmission device, for example via a wireless Bluetooth connection or the like to a computer, a cell phone or the like, by which the user can detect the distribution of the fluorescent microorganisms on his teeth and initiate appropriate steps, especially regarding the need for irradiation using the primary and/or the optional secondary irradiation light source, or also regarding the respective irradiation duration. Fluorescence radiation and transmitted radiation can further be detected by the eye, by CCD cameras such as present in cell phones, and by other photon detectors such as a light diode or a photomultiplier.

According to another preferred embodiment of the present invention, the excitation light source, the primary irradiation light source and/or the optional secondary irradiation light source may comprise at least one of a light-emitting diode (LED), an organic light-emitting diode (OLED) and/or a laser. Accordingly, the handheld device according to the invention may comprise radiation sources in the form of the excitation light source, the primary irradiation light source and/or the optional secondary irradiation light source, preferably high divergence LEDs. For example, the LEDs may be arranged in the form of an array on or in the handheld device. With the divergence preferably being high, it can advantageously be achieved that efficient focusing of the light radiation on the retina of the respective eye a user will not be performed when incorrectly applying the irradiation light sources of the hand-held device to the eyes, and accordingly possible damage of the eye region in case of incorrect application of the hand-held device is not likely to occur. By using miniaturized light sources, especially LEDs, power consumption during irradiation can be kept low, and all advantages usually achieved by LEDs, such as the longevity thereof, etc., also apply to the hand-held device according to the invention. In addition, the device may be provided with a pressure sensor which is used to switch to higher light intensity not before a pressure will be applied which corresponds to the typical pressure of a toothbrush onto the tooth material during toothbrushing. Preferably, the pressure sensor is arranged in the toothbrush head.

The hand-held device according to the present invention preferably is a tooth cleaning device for use during daily dental hygiene, such as a manual toothbrush for manual theeth cleaning or an automatic or electric toothbrush for the automatic teeth cleaning, wherein the excitation light source, the primary irradiation light source and/or the optional secondary irradiation light source may be located in a brush head of the tooth cleaning device and radiation is performed in the bristle direction, that is, in the direction of the bristles extending away from the hand-held device. More specifically, the handheld device may be a manually operated toothbrush, also referred to as a light toothbrush, in which the brush head of the toothbrush may be provided with an interchangeable bristle carrier, or the handheld device may be an electrically operated toothbrush, in which the brush head, which can be set in rotation and/or oscillation, can be replaceable, and wherein a power supply for the electrical drive of the brush head and a power supply for the excitation light source, the primary irradiation light source and/or the secondary irradiation light source can be provided by the same power source.

Alternatively, implementation of the hand-held device in the manner of a temporarily insertable dental plastic splint is conceivable, such as a customized deep-drawn dental plastic splint (covering over teeth and jaw made of translucent plastic), such as an upper head splint or a lower jaw splint, which can be placed over the teeth of a user's jaw, wherein the power source, switch and LED radiation sources can be integrated into the plastic body and positioned at any desired locations on the plastic support. As another, but not preferred alternative, implementation of the hand-held device for the excitation and irradiation of pathogenic microorganisms in the throat without any cleaning function is also conceivable.

Generally, the above-mentioned excitation light source can be used at low intensity for bacterial detection in the throat by fluorescence activation of porphyrins in the pathogenic microorganisms and at higher intensity for inactivation of pathogenic surface microorganisms. Furthermore, the primary irradiation light source can serve to inactivate deeper pathogenic microorganisms or as a transmission source for transillumination for detection and progression of sinusitis. In addition, the optional secondary irradiation light source may serve to further inactivate pathogenic microorganisms, quasi as a supplemental irradiation in a secondary radiation region. Accordingly, the excitation light source may emit violet light at 405 nm, the primary irradiation light source may emit red light at 633 nm, and the secondary irradiation light source may emit yellow or green light at 505 nm, wherein the violet light and the red light may serve for microorganism detection in the throat region, and the violet light can serve for fluorescence activation of porphyrins in the microorganisms, i.e., it can be used at lower intensity for fluorescence excitation and at higher intensity for inactivation of superficial microorganisms. The red light can also allow detection of inflammatory changes, such as sinusitis, and tracking of progression thereof through transillumination. The green light, similar to the red light at higher intensity, can be used to inactivate microorganisms located in deeper tissue layers. However, higher irradiation light intensities in a range of 10 mW/cm² to 100 mW/cm² are emitted by the light toothbrush according to the invention only when the pressure sensor detects a contact pressure, which signals that the light toothbrush has come into contact with either the tooth surface or the tissue in the mouth and throat for tooth cleaning. Such pressure sensor can be in the form of a piezoresistive or piezoelectric pressure sensor, or also in the form of a Hall-effect pressure sensor, whereby any type of commercially available pressure sensor can be used, which can be installed in the light toothbrush according to the invention due to its size and/or mode of operation. Advantageously, the pressure sensor is located in the toothbrush head adjacent to the brushes.

According to the invention, the red radiation around 630 nm of the hand-held device according to the invention furthermore is to be used for transillumination, wherein the red radiation at 630 nm is in the optical window of biological tissue and is characterized by a high light penetration depth of several millimeters. The light penetration depth usually is the value where the light intensity has dropped to about 37% of the initial intensity. Many red photons can therefore travel distances of several centimeters. Thus, red radiation can also be transmitted through the skin. The red transilluminated radiation can easily be detected by eye. In addition, any cell phone equipped with a normal CCD camera, for example, or other photon detectors, such as light diodes and photomultipliers (secondary electron multipliers), is also suitable for image acquisition. Thus, sinusitis can be detected and progress thereof can be observed and documented. The handheld device according to the invention and use thereof are particularly suitable for children and adolescents. Blood absorbs in the range around 400 nm (Soret band) and in the range 540 nm to 580 nm. The endogenous absorber protoporphyrin absorbs around 405 nm, at 505 nm, 540 nm, 573 nm and 635 nm. In order to obtain penetration depths higher than at 400 nm and to be absorbed by, for example, the protoporphyrin PP IX, light in the range around 505 nm (green) and 633 nm (red) should be used according to the invention. This could also be used to inactivate bacteria in the tissue depth, especially in soft tissue. Radiation around 405 nm is used for surface excitation of fluorescence and for inactivation of bacteria on the surface.

According to a preferred embodiment of the present invention, the handheld device comprises an energy source integrated for supplying power to the excitation light source, the primary irradiation light source and/or the secondary irradiation light source, such as, for example, a battery or an accumulator, wherein the integrated energy source, when being implemented as an accumulator, can wirelessly be recharged.

In the following description of the preferred embodiments of the invention, equal or similar components and elements are designated using equal or similar reference numbers, and repeated description of these components or elements in individual cases will be omitted. The figures only schematically represent the subject matter of the invention.

The invention is explained in more detail below while making reference to the figures described below, wherein:

FIG. 1 shows the survival rate of various bacterial strains cultivated in large-scale following single irradiation using 632.8-nm-radiation;

FIG. 2 shows the survival rate of microbiological samples from periodontitis patients following single irradiation using 632.8-nm-radiation;

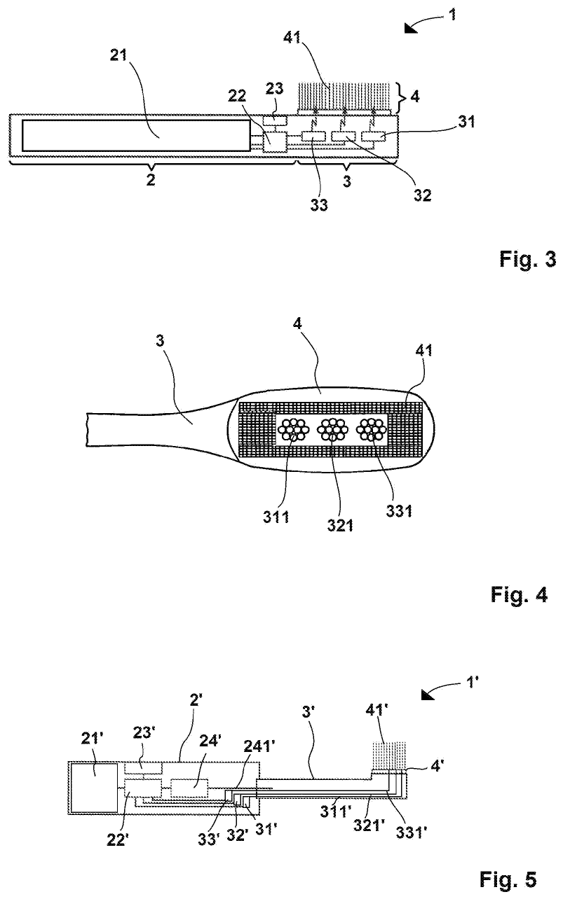

FIG. 3 shows a schematic sectional view of a light toothbrush according to the invention without electrically controlled brush movement for mechanical and optical every-day dental hygiene;

FIG. 4 shows a schematic representation of a brush head of the light toothbrush of FIG. 3 according to the invention; and

FIG. 5 shows a schematic sectional view of a light toothbrush according to the invention with electrically controlled brush movement for mechanical and optical every-day dental hygiene.

According to a preferred embodiment, the hand-held device according to the invention for stimulating and irradiating pathogenic microorganisms in the mouth and throat is implemented as a manually operated mechanical toothbrush 1, for the mechanical and optical every-day dental hygiene by mechanical plaque reduction and photodynamic inactivation of microorganisms in the mouth and throat, as exemplarily shown in FIG. 3. Accordingly, the toothbrush 1 according to the invention can also be referred to as a light toothbrush 1, as cleaning effect is achieved not only by mechanical cleaning but, in addition, by light emission. Accordingly, the toothbrush 1 essentially comprises a toothbrush shaft 2, a toothbrush head 3 and a removable bristle carrier 4. A power or energy source 21 is integrated in the toothbrush shaft 2, for example in the form of a rechargeable battery which can wirelessly be recharged, such as by non-wire electromagnetic fields. Alternatively, the energy source 21 may be in the form of a replaceable energy source, for example in the form of a replaceable AA battery, or also in the form of a miniaturized energy source, such as a replaceable or also rechargeable button cell, to generally reduce the weight of the toothbrush 1. A circuit logic 22 is further arranged in the toothbrush shaft 2, for example in the form of a computer chip or the like, which controls a power distribution to light sources 31, 32, 33, wherein an interface in the form of a push button or switch 23 is further provided in the toothbrush shaft 2, through which a user can interact with the computer chip, for example to control the actuation of the light sources 31, 32, 33. In this context, the switch 23 may also be provided in the form of a touch screen on which a power supply for the individual light sources 31, 32, 33, for example, can be displayed. Furthermore, the light toothbrush 1 comprises a pressure sensor (not shown) which detects a contact pressure of the bristle carrier 4 or the toothbrush head 3 onto the targeted surface, comparing it with the usual contact pressure during tooth brushing, thereby detecting whether the bristle carrier 4 or the toothbrush head 3 is in contact with a tooth surface or the like in the mouth and throat or not, by means of the circuit logic 22. For safety reasons, the circuit logic 22 can control and increase a light intensity of the light sources 31, 32, 33 accordingly to a range of 10 mW/cm² to 100 mW/cm² only when this detection is performed, whereby, for example, a light intensity value hazardous to the eyes may be attained.

The light sources 31, 32, 33 are arranged in the toothbrush head 3 of the toothbrush 1 and, in the present example embodiment, comprise an excitation light source 31, a primary irradiation light source 32 and optionally a secondary irradiation light source 33, which are arranged in a top-down-arrangement during operation of the toothbrush 1, or from right to left in FIG. 3, i.e. in the form of a traffic light arrangement. In the present embodiment, the light sources 31, 32, 33 each comprise an LED or an arrangement of a plurality of LEDs, such as a so-called LED array, wherein the LED light sources 31, 32, 33 can be controlled by actuating the switch 23. Alternatively, the light sources each may also comprise an OLED or an array of OLEDs, or one or more lasers, which may be driven by actuating the switch 23. As indicated by zig-zag arrows in FIG. 3, the light sources 31, 32, 33 emit radiation which is emitted divergently outwards through the bristle carrier 4 as well as the bristles 41 thereon. This can be implemented without making use of additional optical components.

In the present example embodiment, however, the radiation transmission is implemented by optical fibers 311, 321, 331, wherein an optical fiber bundle 311 directs the excitation radiation in the range of 400 nm to 410 nm, preferably 405 nm, from the excitation light source 31 to the outside through the bristles 41, an optical fiber bundle 321 directs the irradiation radiation of the primary irradiation light source in the range of 630 nm to 700 nm, preferably 633 nm, from the primary irradiation light source 32 to the outside through the bristles 41, and an optional light fiber bundle 331 directs the optional irradiation radiation of the optional secondary irradiation light source in the range of 490 nm to 560 nm, preferably 505 nm, from the optional secondary irradiation light source 33 to the outside through the bristles 41. In the present embodiment, the emitting ends of the optical fiber bundles 311, 321, 331 are arranged in a region of the toothbrush head 3 which is kept free of bristles 41, as shown in FIG. 4. Alternatively, however, the bristles 41 may cover the entire surface of the bristle carrier 4, in which case the bristles 41 themselves may serve as an extension of the light fiber bundles 311, 321, 331, i.e. may be designed as light fibers. Basically, the hand-held device according to the invention can also be implemented in the form of a manually operated mechanical toothbrush comprising a non-removable bristle carrier, or in the form of an electrically operated toothbrush comprising a removable toothbrush head, in which case the light sources can be arranged in the fixed toothbrush shaft, only requiring a radiation guide outwards decouplable from the light sources.

Furthermore, in the present embodiment, the toothbrush 1 may comprise at least one light detection device (not shown) arranged for detecting an intrinsic fluorescence radiation of the pathogenic microorganisms, so that the user is not required to rely on the visual detection of all intrinsically fluorescent microorganisms, but may pass this task to the light detection device. Accordingly, the light detection device can detect the intrinsic fluorescence of the excited microorganisms and output a corresponding detection signal, for example by means of a haptic or acoustic feedback to the user, wherein the strength of the feedback can reflect the intensity of the intrinsic fluorescence, or also as a visual display on the switch 23 designed as a touch screen. Furthermore, the light detection device can transmit the detection signal, i.e. the distribution of the fluorescent microorganisms at the end of the user's teeth, to the outside by means of a transmission device (not shown), for example via a wireless Bluetooth connection or the like to a computer, a cell phone or the like, by which the user can recognize the distribution of the fluorescent microorganisms on his teeth and take appropriate steps, especially with regard to the required irradiation by the primary and secondary irradiation light sources 32, 33, the intensity thereof, or also with regard to the respective irradiation duration.

According to another preferred embodiment as shown in FIG. 5, the hand-held device according to the invention can also be integrated into an electrically operated toothbrush 1′ or also as an automatically operated toothbrush. In this case, the electric toothbrush 1′ consists of a toothbrush shaft or toothbrush base body 2′, an exchangeable toothbrush head 3′ integrated with a bristle carrier 4′. A power or energy source 21′ is integrated into the toothbrush shaft 2′, for example in the form of a rechargeable battery that can wirelessly be charged, such as by non-wire electromagnetic fields. The electric light toothbrush 1′ also in turn comprises a pressure sensor (not shown) that detects a contact pressure of the bristle carrier 4′ or the toothbrush head 3′, whereby it is possible to detect whether the bristle carrier 4′ or the toothbrush head 3′ is in contact with a tooth surface or the like in the mouth and throat or not. Furthermore, a circuit logic 22′ is arranged in the toothbrush shaft 2′, for example in the form of a computer chip or the like, which regulates a power distribution to light sources 31′, 32′, 33′ also arranged in the toothbrush shaft 2′, wherein furthermore an interface in the form of a push button or switch 23′ is provided in the toothbrush shaft 2′, by which a user can interact with the computer chip, for example to control activation of the light sources 31′, 32′, 33′. In this context, the switch 23′ can also be provided in the form of a touch screen on which a power supply for the individual light sources 31′, 32′, 33′ can, for example, be displayed. The light sources 31′, 32′, 33′ are located in the toothbrush head 3 of the toothbrush 1′ and, in the present example embodiment, comprise an excitation light source 31′, a primary irradiation light source 32′ and an optional secondary irradiation light source 33′, which are arranged in a top-down arrangement during operation of the toothbrush 1′, or from right to left in FIG. 5. The light sources 31′, 32′, 33′ in the present embodiment each comprise an LED or an arrangement of a plurality of LEDs, such as a so-called LED array, wherein the LED light sources 31′, 32′, 33′ can be controlled by actuating the switch 23′. Furthermore, in the present embodiment, a motor 24′ is provided for operating a drive shaft 241′, which can also be operated by the power source 21′ and actuated by the switch 23′, the drive shaft 241′ being provided for exerting the mechanical movement of the attachable brush head 3′.

As it is shown in FIG. 5, the LED light sources 31, 32, 33 emit radiation which is guided through optical fibers 311′, 321′, 331′ to the bristle carrier 4′ and, through the bristles 41′ thereof, is divergently emitted outwards. This radiation guide can alternatively be implemented by one or more mirrors as alternative light guiding paths without optical fibers. In the present example embodiment, the beam transmission is performed by the optical fibers 311′, 321′, 331′, wherein the optical fiber bundle 311′ directs the excitation radiation in the range of 400 nm to 410 nm, preferably 405 nm, from the excitation light source 31′ to through the bristles 41′, the optical fiber bundle 321′ directs the irradiation radiation in the range of 630 nm to 700 nm, preferably 633 nm, from the primary irradiation light source 32′ to the outside through the bristles 41′, and the optional light fiber bundle 331′ directs the optional irradiation radiation in the range of 490 nm to 560 nm, preferably 505 nm, from the optional secondary irradiation light source 33′ to the outside through the bristles 41′, wherein a radiation guide is outwards decouplable from the light sources 31′, 32′, 33′ to enable replacement of the brush head 3′.

The technical features of the embodiments described above are not limited to the particular embodiment described and accordingly are interchangeable.

What is claimed:

1. A hand-held device for excitation and irradiation of pathogenic microorganisms in the mouth and throat, comprising
at least one excitation light source in the short-wave visible spectral range for auto-fluorescence excitation and irradiation of the pathogenic microorganisms,
at least one primary irradiation light source in the red spectral range for primary irradiation of the pathogenic microorganisms and for transillumination, and
optionally at least one secondary irradiation light source in the visible spectral range from 450 nm to 600 nm, wherein
the emitted radiation of the irradiation light sources each have spectral components which can be absorbed by endogenous porphyrins produced by the pathogenic microorganisms, whereby fluorescence formation and inactivation of the pathogenic microorganisms occurs, and
the hand-held device further comprises a pressure sensor which, upon detection of a pressure of the hand-held device onto an affected area, increases an irradiation light intensity to values in a range of 10 mW/cm2 to 100 mW/cm2 to achieve inactivation of the pathogenic microorganisms.

2. The handheld device according to claim 1, wherein the at least one excitation light source for auto-fluorescence excitation and irradiation of the pathogenic microorganisms emits an excitation radiation corresponding to an absorption maximum of porphyrins.

3. The handheld device according to claim 2, wherein the at least one excitation light source for auto-fluorescence excitation and irradiation of the pathogenic microorganisms emits an excitation radiation in a range of 400 nm to 410 nm.

4. The hand-held device according to claim 1, wherein the at least one primary irradiation has a wavelength component in a range from 630 nm to 700 nm.

5. The hand-held device according to claim 1, wherein when present, the at least one optional secondary irradiation has a wavelength component in a range from 490 nm to 560 nm.

6. The hand-held device according to claim 1, wherein when present, emission of the optional at least secondary irradiation light source radiation is performed subsequently to emission of the at least one primary irradiation light source radiation, and wherein emission of the at least one primary irradiation light source radiation is performed subsequently to emission of the at least one excitation light source radiation.

7. The handheld device according to claim 1, wherein the handheld device further comprises at least one light detection device for detecting an intrinsic fluorescence radiation of the pathogenic microorganisms.

8. The handheld device according to claim 1, wherein the at least one excitation light source, the at least one primary irradiation light source and/or the optional at least one secondary irradiation light source comprises at least one of a light-emitting diode LED, an organic light-emitting diode OLED and/or a laser.

9. The hand-held device according to claim 1, wherein the at least one excitation light source, at low intensity, is for bacterial detection in the throat by fluorescence activation of porphyrins in the pathogenic microorganisms and, at higher intensity, for inactivation of surface pathogenic microorganisms, the at least one primary irradiation light source is for inactivation of deeper located pathogenic microorganisms and as a transmission source by transillumination for the detection and follow-up of sinusitis, and the optional at least one secondary irradiation light source is for inactivation of deeper located pathogenic microorganisms.

10. The hand-held device according to claim 1, wherein the hand-held device is a manually or electrically operated toothbrush.

11. A method of inactivating a pathogenic microorganism within a users' oral cavity comprising:
applying light of a first wavelength to the pathogenic microorganism from an excitation light source to a tissue infected with the pathogenic microorganism; and
applying light of a second wavelength to the pathogenic microorganism from an irradiation light source to the tissue infected with the pathogenic microorganism;
wherein the application of the light of first and second wavelengths is sufficient to inactivate the pathogenic microorganism.

12. The method of claim 11, wherein the first wavelength of light is different from the second wavelength of light.

13. The method of claim 12, wherein the first wavelength of light is in the short-wave visible spectral range and the second wavelength of light is in the red spectral range.

14. The method of claim 11, further comprising applying either the first wavelength of light or the second wavelength of light, or both, to the oral cavity tissue at multiple intensities.

15. The method of claim 14, wherein the second wavelength of light is applied at multiple intensities.

16. The method of claim 15, wherein the first wavelength of light is applied at a single intensity.

17. The method of claim 16, wherein the first wavelength of light penetrates the oral tissue surface to a depth of about 1 mm or less.

18. The method of claim 16, wherein a first intensity of the second wavelength is about 10 mW/cm2 and a second intensity of the second wavelength is about 100 mW/cm2.

19. The method of claim 18, wherein the second wavelength of light is applied at about 100 mW/cm2 of intensity and 360 J/cm2 of energy density.

20. The method of claim 14, further comprising initiating the second intensity upon application of pressure between a device containing the irradiation light source and the microorganism.
